(19) United States
(12) Patent Application Publication
(10) Pub. No.: US 2010/0143506 A1
(43) Pub. Date: Jun. 10, 2010

(54) APPARATUS AND METHOD FOR TREATMENT OF MICROORGANISMS DURING SUGAR PRODUCTION AND SUGAR-BASED FERMENTATION PROCESSES

(76) Inventor: Allen Michael Ziegler, Littleton, CO (US)

Correspondence Address: MCANDREWS HELD & MALLOY, LTD, 500 WEST MADISON STREET, SUITE 3400, CHICAGO, IL, 60661, US

(21) Appl. No.: 12/625,184

(22) Filed: Nov. 24, 2009

Related U.S. Application Data
(60) Provisional application No. 61/117,510, filed on Nov. 24, 2008.

(51) Int. Cl. A61K 33/00; C12M 1/00; C12N 1/16

(52) U.S. Cl. 424/722; 435/283.1; 435/255.1; 435/289.1

(57) ABSTRACT

A method of reducing undesirable microorganism concentration in an aqueous fluid stream employed in a sugar production process or a sugar-based fermentation production process includes (a) generating ClO₂ gas, (b) dissolving the ClO₂ gas to form a ClO₂ solution, and (c) introducing an aqueous ClO₂ solution into the aqueous fluid stream. Another method includes introducing ClO₂ having an efficiency as ClO₂ of at least about 90% into the aqueous fluid stream. An apparatus for reducing undesirable microorganism concentration comprises a ClO₂ generator fluidly connected to a batch tank, fluidly connected to a sugar production vessel or sugar-based fermentation vessel.

### CROSS-REFERENCE TO RELATED APPLICATION(S)

This application claims priority benefits from U.S. Provisional Patent Application Ser. No. 61/117,510, filed Nov. 24, 2008, entitled “Apparatus And Method For Treatment Of Microorganisms During Sugar Production and Sugar-Based Fermentation Processes”. The '510 applications is hereby incorporated by reference herein in its entirety.

### FIELD OF THE INVENTION

Generally, the technical field involves sugar production and sugar-based fermentation processes. Specifically, it is a method of reducing the concentration of undesirable microorganisms during sugar production and/or sugar-based fermentation processes while simultaneously encouraging propagation and/or conditioning of desirable microorganisms and increasing the efficiency of desirable microorganisms during sugar-based fermentation processes.

### BACKGROUND OF THE INVENTION

Sugars are a class of water-soluble crystalline carbohydrates. Examples of sugars include sucrose, fructose, glucose and lactose. Sugars have a characteristically sweet taste and are commonly used as sweeteners in many foods, drinks and medicines.

Sugars can also be used in fermentation processes. In these processes microorganisms such as yeast, fungi and bacteria convert the sugars into cellular energy and produce aliphatic alcohols as by-products. These fermentation processes can be used to produce items such as industrial grade ethanol, distilled spirits, beer, wine, pharmaceuticals and nutraceuticals (foodstuff that provides health benefits, such as fortified foods and dietary supplements).

There is a large market for sugars for human consumption and/or use in fermentation processes. World sugar consumption for the 2009/2010 marketing year is forecast at 153.7 million tons by the United States Department of Agriculture, Foreign Agricultural Service. See http://www.fas.usda.gov.

Sugars for consumption and/or fermentation processes can be derived from a number of sources. Sugars primarily come from sugar cane and from sugar beets, but also appear in fruit, honey, sorghum, sugar maple and in many other sources. The starting material goes through a treatment process which produces an extraction which can then be treated for sale as consumable sugar or sent into a fermentation process. It is typical for a single facility to treat the starting materials and then alternate between sugar production and production of a fermentation product, such as ethanol.

At some point during the starting material preparation process, the sugar production process and/or the overall fermentation process the stream of material being treated can become contaminated with bacteria or other undesirable microorganisms. This can occur in one of the many vessels used in the starting material preparation process, the sugar production process and/or the overall fermentation process

Bacterial or microbial contamination in products intended for human consumption, such as the raw sugar, is undesirable because of health concerns. Contamination also reduces the fermentation product yield. This occurs in three main ways. First, the sugars that could be available for the desirable producing microorganisms to produce alcohol are consumed by the bacteria or other undesirable microorganisms and diverted from alcohol production. In addition to reducing yield, the end products of bacterial metabolism, such as lactic acid and acetic acid, inhibit growth, fermentation and/or respiration of the desirable producing microorganisms, which results in less efficient production by those microorganisms. Finally, the bacteria or other undesirable microorganisms compete with the desirable producing microorganisms for nutrients other than sugar.

After the stream or vessel has become contaminated with bacteria or other undesirable microorganisms, those bacteria or other microorganisms can grow much more rapidly than the desirable producing microorganisms. The bacteria or other microorganisms compete with the desirable producing microorganisms for fermentable sugars and retard the desired bio-chemical reaction resulting in a lower product yield. Bacteria also produce unwanted chemical by-products, which can cause spoilage of entire fermentation batches. Removing these bacteria or other undesirable microorganisms allows the desirable producing microorganisms to thrive, which results in higher efficiency.

As little as a one percent decrease in ethanol yield is highly significant to the fuel ethanol industry. In larger facilities, such a decrease in efficiency will reduce income from 1 million to 3 million dollars per year.

Some previous methods of reducing bacteria or other undesirable microorganisms during fermentation processes apply heat to or lower the pH of the fermentation solution. However, these processes are not entirely effective in retarding bacterial growth. Furthermore, the desirable producing microorganisms, while surviving, are stressed and not as vigorous or healthy. Thus, the desirable producing microorganisms do not perform as well.

The predominant trend in the ethanol industry is to reduce the pH of the mash to less than 4.5 at the start of fermentation. Lowering the pH of the mash reduces the population of some species of bacteria. However it is much less effective in reducing problematic bacteria, such as lactic-acid producing bacteria, and is generally not effective for wild yeast and molds. It also significantly reduces ethanol yield by stressing the desirable producing microorganisms.

Another current method involves the addition of antibiotics to the fermentation process to neutralize bacteria. This method has a number of problems. Antibiotics are expensive and can add greatly to the costs of large-scale production. Improved technology that refines and improves the efficiency of existing techniques would be of considerable value to the industry. Moreover, antibiotics are not effective against all strains of bacteria, such as antibiotic-resistant strains of bacteria. Overuse of antibiotics can lead to the creation of additional variants of antibiotic-resistant strains of bacteria. Antibiotic residues and establishment of antibiotic-resistant strains is a global issue. These concerns may lead to future regulatory action against the use of antibiotics.

In addition, there are other issues to consider when using antibiotics. Calculating the correct dosage of antibiotic can be a daunting task. Even after dosages have been determined, mixtures of antibiotics should be constantly or at least frequently balanced and changed in order to avoid single uses that will lead to antibiotic-resistant strains. Sometimes the effective amount of antibiotic cannot be added to the fermentation mixture. For example, utilizing over 6 mg/L of Virginiamycin will suppress fermentation but over 25 mg/L is required to inhibit grown of Weisella confusa, an emerging problematic bacteria strain.

Another approach involves washing the desirable producing microorganisms with phosphoric acid. This method does not effectively kill bacteria and other microorganisms. It can also stress the desirable producing microorganisms, thereby lowering their efficiency.

Yet another method is to use heat or harsh chemicals and sterilize process equipment between batches. However this method is only effective when equipment is not in use. It is ineffective at killing bacteria and other microorganisms within the mixture during production.

Chlorine dioxide (ClO₂) has many industrial and municipal uses. When produced and handled properly, ClO₂ is an effective and powerful biocide, disinfectant and oxidizer. ClO₂ has been used as a disinfectant in the food and beverage industries, wastewater treatment, industrial water treatment, cleaning and disinfections of medical wastes, textile bleaching, odor control for the rendering industry, circuit board cleansing in the electronics industry, and uses in the oil and gas industry. It is an effective biocide at low concentrations and over a wide pH range. ClO₂ is desirable because when it reacts with an organism in water, it reduces to chlorite ion and then to chloride, which studies to date have shown does not pose a significant adverse risk to human health. ClO₂ is, however, unstable in the gas phase and will readily undergo decomposition into chlorine gas (Cl₂), oxygen gas (O₂), and heat.

Previously, brewers added an aqueous 2-6% by weight sodium chlorite solution, otherwise known as stabilized chlorine dioxide, to their fermentation batches in an attempt to kill bacteria and other microorganisms. When sodium chlorite reacts in an acidic environment it can form ClO₂. The ClO₂ added using this method was not substantially pure, which made it difficult to ascertain the amount added or control that amount with precision. If the amount is not precisely maintained, the ClO₂ can kill the desirable producing microorganisms. If this occurs, the addition of ClO₂ will not result in more efficient production. This method is also not effective at a neutral or basic pH level.

Generated or substantially pure ClO₂ has been found to be effective in treating microorganisms during conditioning, propagation and fermentation procedures. This is discussed in Applicants' related U.S. application Ser. No. 11/626,172, filed Jan. 23, 2007, which relates to and claims priority benefits from U.S. Provisional Patent Application Ser. No. 60/775,615, filed Feb. 22, 2006, entitled “Apparatus And Method For Treatment Of Yeast During Propagation, Conditioning And Fermentation.” The '172 and '615 applications are hereby incorporated by reference herein in their entirety

### SUMMARY OF THE INVENTION

The current disclosure relates to a method for reducing the concentration of bacteria and other undesirable microorganisms during sugar production and sugar-based fermentation processes while simultaneously encouraging propagation and/or conditioning of desirable microorganisms and increasing the efficiency of those desirable microorganisms in the sugar-based fermentation processes and an apparatus for carrying out this method.

Certain embodiments of the current method comprise the steps of:
(a) employing an aqueous fluid stream in a sugar production process;
(b) generating ClO₂ gas;
(c) dissolving the ClO₂ gas to form a ClO₂ solution;
(d) introducing an aqueous ClO₂ solution into the stream.

Certain embodiments of the current method comprise the steps of:
(a) employing an aqueous fluid stream in a sugar production process; and
(b) introducing ClO₂ having an efficiency as ClO₂ of at least 90% into the stream.

Certain embodiments of the present apparatus comprise:
(a) a ClO₂ generator comprising an inlet for introducing at least one chlorine-containing feed chemical and an outlet for exhausting a ClO₂ gas stream from the generator;
(b) a batch tank fluidly connected to the ClO₂ generator outlet, the batch tank receiving the ClO₂ gas stream from the ClO₂ generator outlet, the batch tank comprising an inlet for introducing a second water stream and an outlet for exhausting an aqueous ClO₂ solution from the batch tank;
(c) a vessel utilized in a sugar-production process, the vessel fluidly connected to the batch tank;
   wherein introducing the ClO₂ solution from the batch tank to the vessel reduces undesirable microorganism concentration in the vessel.

Certain embodiments of the current method comprise the steps of:
(a) introducing a quantity of fermentable sugar to the stream;
(b) introducing a quantity of yeast to the stream;
(c) generating ClO₂ gas;
(d) dissolving the ClO₂ gas to faun a ClO₂ solution;
(e) introducing an aqueous ClO₂ solution into the stream.

Certain embodiments of the current method comprise the steps of:
(a) introducing a quantity of fermentable sugar to the stream;
(b) introducing a quantity of yeast to the stream; and
(c) introducing ClO₂ having an efficiency as ClO₂ of at least 90% into the stream.

Certain embodiments of the present apparatus comprise:
(a) a ClO₂ generator comprising an inlet for introducing at least one chlorine-containing feed chemical and an outlet for exhausting a ClO₂ gas stream from the generator;
(b) a batch tank fluidly connected to the ClO₂ generator outlet, the batch tank receiving the ClO₂ gas stream from the ClO₂ generator outlet, the batch tank comprising an inlet for introducing a second water stream and an outlet for exhausting an aqueous ClO₂ solution from the batch tank;
(c) a vessel for containing an aqueous yeast solution, the vessel fluidly connected to the batch tank;
   wherein introducing the ClO₂ solution from the batch tank to the vessel promotes propagation of yeast present in the vessel

These and other features of the present technique are discussed or apparent in the following detailed description.

### BRIEF DESCRIPTION OF THE DRAWINGS

FIG. 1 is a flow diagram of the process for production of sugar and sugar-based fermentation products.

FIG. 2 is a schematic of combined sugar and sugar-based fermentation equipment with an integrated ClO₂ system in accordance with one embodiment.

The foregoing summary, as well as the following detailed description of certain embodiments of the present technique, will be better understood when read in conjunction with the appended drawings. For the purpose of illustrating the technique, certain embodiments are shown in the drawings. It should be understood, however, that the present technique is not limited to the arrangements and instrumentalities shown in the attached drawings.

### DETAILED DESCRIPTION OF PREFERRED EMBODIMENT(S)

The current disclosure relates to a method for reducing the concentration of bacteria and other undesirable microorganisms during sugar production and/or sugar-based fermentation processes while simultaneously encouraging propagation and/or conditioning of desirable microorganisms and increasing the efficiency of those desirable microorganisms in the sugar-based fermentation processes and an apparatus for carrying out this method.

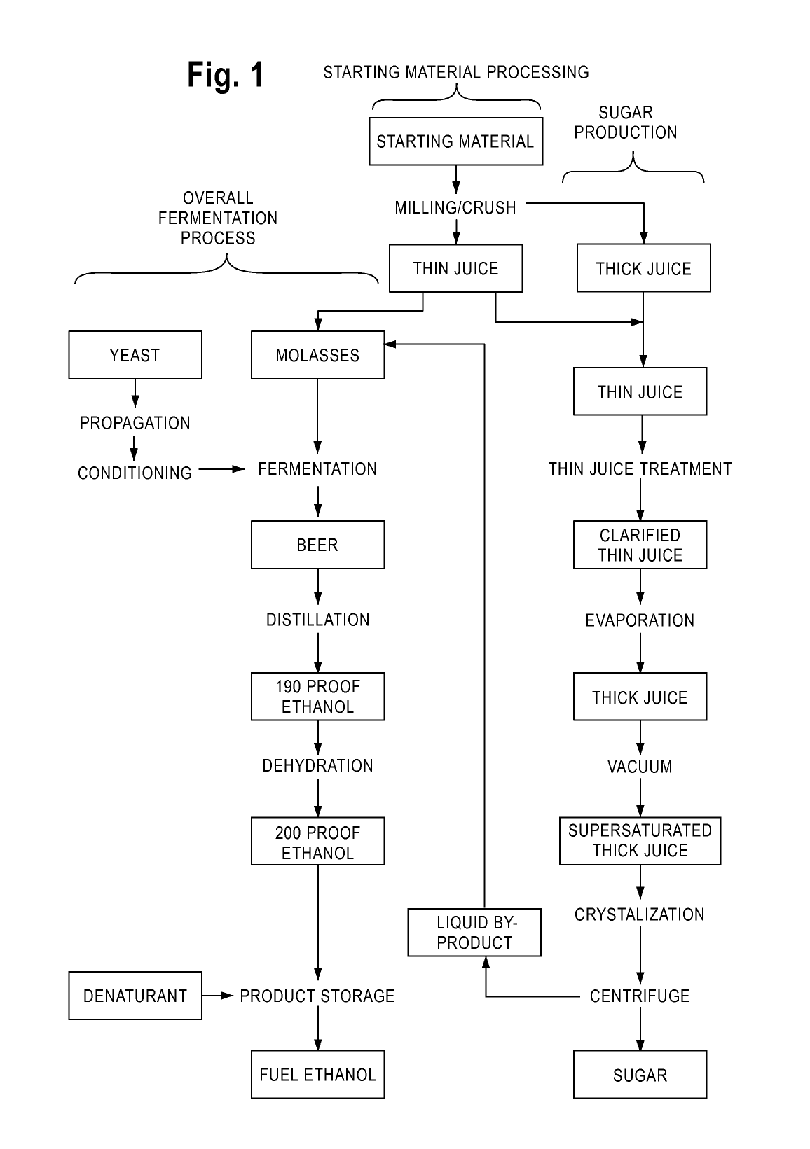

FIG. 1 illustrates the process for production of sugar and/or a sugar-based fermentation product. Production of sugar and production of a sugar-based fermentation product begin in a similar manner. At a certain point the processes diverge to obtain different end products. It is typical for a single facility to alternate between sugar production and production of a sugar-based fermentation product. For this reason the current disclosure examines the two processes together.

The production of consumable sugar and fuel ethanol by yeast fermentation from sugarcane is used as an example. However, this is merely one illustration and should not be understood as a limitation. Other fermentation products could include distilled spirits, beer, wine, pharmaceuticals, pharmaceutical intermediates, baking products, nutraceuticals (foodstuff that provides health benefits, such as fortified foods and dietary supplements), nutraceutical intermediates and enzymes. Other fermenting microorganisms could also be substituted, such as fungi and bacteria. Other sugar sources could also be used, such as sugar beets or citrus pulp.

Both the sugar production and sugar-based fermentation processes begin with the preparation of a starting material. Examples of possible starting materials include sugar cane, sugar beets, fruit (such as citrus pulp), honey, sorghum, and sugar maple. The starting material undergoes processing to extract the sugar. This processing can involve washing the starting material and cutting it into small pieces. These pieces can then be mixed with water and repeatedly crushed between rollers. This crushing or milling extracts a liquid containing about 15-20 percent by weight sucrose from the starting material. This liquid is sometimes called thin juice or sugar juice. The thin juice begins to ferment almost immediately. A solid also remains. The solid can be used for animal feed, in paper manufacture, or burned as fuel.

Current practice is to add a non-oxidizing biocide at milling and/or to the thin juice to control unwanted microbiology. Example of non-oxidizing biocides include Glutaraldehyde, 1,5 pentanedial Use Rat (1-100 ppm), 2,2-dibromo-3-nitrilopropionamide (1-30 ppm), 5-chloro-N-methylisothiazolone & N-methylisothiazolone (Typically referred to as Isothiazolone) (1-30 ppm), 1,2-benzisothiazolone (1-30 ppm), Thiocarbamates, Potassium N-dimethyldithiocarbamate (1-30 ppm), Poly-Quats, Poly[oxyethylene(dimethylinimio)ethylene-(dimethylinimio)ethylene dichloride] (1-30 ppm). Oxidizing biocides were also used. Examples of oxidizing biocides include bromine, sodium hypochlorite and calcium hypochlorite. The current technology can be used in lieu of or in addition to these biocides to control unwanted microbiology.

The thin juice can then undergo further treatment. The pH of the thin juice can be adjusted. This is sometimes done using lime. This adjustment arrests sucrose's decay into glucose and fructose, and precipitates out some impurities. The mixture can then be allowed to sit, allowing suspended solids to settle out. This results in a clarified thin juice.

The clarified thin juice can then undergo further treatment in order to produce a consumable sugar product or it can enter a sugar-based fermentation process. To form consumable sugar, the clarified thin juice is then concentrated. This can be done in an evaporator. This evaporation creates a thick syrup that is about 60 percent by weight sucrose. This thick syrup is also known as thick juice. The thick juice is further concentrated until it becomes supersaturated. This can be done under vacuum. The supersaturated thick juice can then be seeded with crystalline sugar. Upon cooling, sugar will crystallize out of the syrup. A centrifuge can then be used to separate the sugar from the remaining by-product liquid. The remaining by-product liquid can then be used in a sugar-based fermentation process.

As discussed above, the clarified thin juice can alternatively enter into a sugar-based fermentation process. The by-product liquid remaining after sugar crystallization could also enter into a sugar-based fermentation process. The sugar source used in a fermentation process (either the thin juice or the by-product liquid) is typically referred to as molasses. There are several different types of molasses. For example, high test molasses is the name for the thin juice removed from milling or crushing sugar cane. Blackstrap molasses is the by-product liquid produced during the milling and crushing of sugar cane for sugar production. Refiners cane is the by-product liquid produced during the milling and crushing of brown sugar to produce white sugar. Beet molasses is the by-product liquid produced during the milling and crushing of sugar beets for sugar production. Citrus molasses is the name for sugar juices extracted from citrus pulp. Unlike carbohydrate-based fermentation processes which contain starch, all sugars in the molasses are present and readily available in a fermentable form. Molasses generally do not require cooking and are present in liquid form.

Microorganisms capable of fermentation will also be added to the molasses. Typically yeast are used in fermentation processes. For this reason, yeast will be addressed in further detail throughout the disclosure. However, it should be understood that other desirable producing microorganisms could also be substituted.

Yeast are fungi that reproduce by budding or fission. One common type of yeast is Saccharomyces cerevisia, the species predominantly used in baking and fermentation. Non-Sacharomyces yeasts, also known as non-conventional yeasts, are naturally occurring yeasts that exhibit properties that differ from conventional yeasts. Non-conventional yeasts are utilized to make a number of commercial products such as amino acids, chemicals, enzymes, food ingredients, proteins, organic acids, nutraceuticals, pharmaceuticals, cosmetics, polyols, sweeteners and vitamins. Some examples of non-conventional yeasts include Kuyberomyces lactis, Yarrowia lipolytica, Hansenula polymorpha and Pichia pastoris. The current methods and apparatus are applicable to intermediates and products of both Sacharomyces and non-conventional yeast.

Most of the yeast used in fuel ethanol plants and other fermentation processes are purchased from manufacturers of specialty yeast. The yeast are manufactured through a propagation process and usually come in one of three forms: yeast slurry, compressed yeast or active dry yeast. Propagation is the first step in the overall fermentation process and involves growing a large quantity of yeast from a small lab culture of yeast. During propagation the yeast are provided with the oxygen, nitrogen, sugars, proteins, lipids and ions that are necessary or desirable for optimal growth through aerobic respiration.

Once at the distillery, the yeast can undergo conditioning. Conditioning is the second step in the overall fermentation process. The objectives of both propagation and conditioning are to deliver a large volume of yeast to the fermentation tank with high viability, high budding and a low level of infection by other microorganisms. However, conditioning is unlike propagation in that it does not involve growing a large quantity from a small lab culture. During conditioning, conditions are provided to re-hydrate the yeast, bring them out of hibernation and allow for maximum anaerobic growth and reproduction.

Following propagation and/or conditioning, the yeast enter the fermentation step of the overall fermentation process. The yeast produce energy by converting the sugars into carbon dioxide and aliphatic alcohols, such as ethanol.

The fermented molasses, now called “beer” now enters the processing steps of the overall fermentation process. First the beer is distilled. This process removes the 190 proof ethanol, a type of alcohol, from the solids in the fermented molasses. After distillation, the alcohol is passed through a dehydration system to remove remaining water. At this point the product is 200 proof ethanol. This ethanol can then be denatured by adding a small amount of denaturant, such as gasoline, to make it unfit for human consumption.

The overall fermentation process can be carried out using batch and continuous methods. The batch process is used for small-scale production. Each batch is completed before a new one begins. The continuous fermentation method is used for large-scale production because it produces a continuous supply without restarting every time. The current method and apparatus are effective for both methods.

Sugar-based ethanol facilities typically recycle yeast. These facilities use a yeast centrifuge and yeast process tanks to remove yeast from completed fermentations for reuse. After two to four months, new yeast can be added to the system to recharge the system with fresh yeast. The current method and apparatus are effective for a facility that recycles yeast.

During the starting material preparation process, the sugar production process and/or the overall fermentation process (including propagation, conditioning, fermentation and processing), the material being treated (for example the starting material, the thin juice, the clarified thin juice, the thick juice, the raw sugar product, the molasses, the yeast slurry, the beer, the product ethanol, the by-product liquid) and/or its containment or transfer vessel can become contaminated with other undesirable microorganisms (such as spoilage bacteria, wild yeast or killer yeast). These microorganisms compete with the yeast for fermentable sugars and retard the desired bio-chemical reaction resulting in a lower product yield. They can also produce unwanted chemical by-products, which can cause spoilage of entire fermentation batches. Wild yeast are a primary concern in the beverage industry because they can cause taste and odor problems with the final product. Killer yeast produce a toxin that is lethal to the desirable alcohol producing yeast.

These undesirable microorganisms can also contaminate the pipelines of a sugar production or fermentation apparatus by forming what is known as a bio-film. The bio-film is made up of a backbone of di-sulfide bonds. Undesirable microorganisms congregate and inhabit the area under the film. Removal of a bio-film results in a cleaner system.

In the current disclosure, the “undesirable” microorganisms intended to be reduced are those that compete for nutrients with the desirable microorganisms, such as yeast that produce fermentation products in the fermentation processes involved here. In this regard, the aqueous ClO₂ solution employed in the present method does not appear to detrimentally affect the growth and viability of desirable, fermentation-promoting microorganisms, but does appear to eliminate or at least suppress the growth of undesirable microorganisms that interfere with the fermentation process. Moreover, the elimination or suppression of undesirable microorganisms appears to have a favorable effect on the growth and viability of desirable microorganisms, for the reasons set forth in the Background section.

Producers of ethanol and sugar attempt to increase the amount of ethanol and sugar produced from a given amount of starting materials. Contamination by undesirable microorganisms lowers the efficiency of yeast making it difficult to attain efficient production. Reducing the concentration of undesirable microorganisms will encourage yeast propagation and/or conditioning and increase yeast efficiency making it possible to attain and exceed these desired levels.

Yeast can withstand and indeed thrive in a ClO₂ environment. However, bacteria, wild yeasts, killer yeasts and molds will succumb to the properties of ClO₂ allowing the producing, desirable yeast to thrive and achieve higher production

ClO₂ solution has many uses in disinfection, bleaching and chemical oxidation. ClO₂ can be added at various points in the starting material preparation process, the raw sugar production process and/or the overall fermentation process to kill unwanted microorganisms and promote growth and survival of the desirable microorganisms. This ClO₂ can be added as an aqueous solution or a gas. The ClO₂ can be added during the starting material preparation process, the raw sugar production process and/or the overall fermentation process. The ClO₂ solution can be added to milling vessels, thick juice treatment vessels, thin juice treatment vessels, vacuum pans, sugar crystallizers, evaporators, transfer lines, yeast recycle tanks, yeast separators, centrifuges, beer wells, cook vessels, fermentation tanks, propagation tanks, conditioning tanks, starter tanks or tanks used during liquefaction. The ClO₂ solution can also be added to the interstage heat exchange system or heat exchangers. In one embodiment the ClO₂ has an efficiency as ClO₂ in the stream of at least about 90%. Adding ClO₂ having a known purity allows for addition of a controlled amount of ClO₂.

Chorine dioxide is a selective oxidizer. It provides microbial efficacy in high organic processes that exceeds that of other antimicrobials. The selectivity of the chlorine dioxide allows for removal of the bio-film discussed above due to its affinity to oxidize di-sulfide bonds before reacting with other constituents. When the di-sulfide bonds that make up the backbone of the bio-film are broken, the film can no longer remain connected to the pipe. Initially when the bio-film is being destroyed more bacteria will be exposed to the process since they tend to inhabit the area under the film. Once the bio-film is removed a cleaner system can be realized.

The chlorine dioxide molecule is also selective when reacting with organics and living matter which allows it to kill bacteria and not affect yeast in a highly organic substrate. Chlorine dioxide has a wide pH range in which it can operate (2-10) which allows for treating processes that would inhibit other disinfectants. Chlorine dioxide also does not react with ammonia, unlike chlorine. This is beneficial to a fermentation system since ammonia is a source of yeast nutrition.

As mentioned above, ClO₂ can be added during the milling/crush of the starting material. Chlorine dioxide can be added in an effective amount. As one example, chlorine dioxide dosages of about 20 to about 80 mg/L can be applied during the milling/crush of the starting material.

ClO₂ can be added to the thin juice. Chlorine dioxide can be added in an effective amount. For example, chlorine dioxide dosages of about 10 to about 50, mg/L, or about 20 to about 80 mg/L can be applied to the thin juice. Application of chlorine dioxide to the thin juice line keeps bio-film from forming in the pipeline and reduces the initial count of bacteria going into the distillery.

The ClO₂ can also be added during the sugar juice treatment steps to either the thin juice or the thick juice. Chlorine dioxide can be added in an effective amount. As one example, chlorine dioxide dosages of about 10 to about 50 mg/L can be applied directly to the thin juice or thick juice.

The ClO₂ can also be added to the evaporators, vacuum pans or crystallizers used during the raw sugar production process. Chlorine dioxide can be added in an effective amount. As one example, chlorine dioxide dosages of about 2 to about 50 mg/L can be applied to the evaporators, vacuum pans or crystallizers.

The ClO₂ can also be added directly into the fermentation mixture. Chlorine dioxide can be added in an effective amount. As one example, chlorine dioxide dosages of about 2 to about 30 mg/L can be applied directly to the fermentation mixture.

Chlorine dioxide can also be added during propagation and/or conditioning. Chlorine dioxide can be added in an effective amount. As one example, chlorine dioxide dosages of about 10 to about 85 mg/L can be added during propagation and/or conditioning. Injection at the yeast propagator (pre-fermenter) prevents bacteria from growing.

Chlorine dioxide can also be added to the desirable microorganism recycle tank. Chlorine dioxide can be added in an effective amount. As one example, chlorine dioxide dosages of about 10 to about 85 mg/L can be added to the desirable microorganism recycle tank.

Chlorine dioxide can also be added to the yeast separator and/or centrifuge. Chlorine dioxide can be added in an effective amount. As one example, chlorine dioxide dosages of about 10 to about 85 mg/L can be added to the yeast separator and/or centrifuge.

Chlorine dioxide can also be added to the beer well. Chlorine dioxide can be added in an effective amount. As one example, chlorine dioxide dosages of about 2 to about 40 mg/L can be added to the beer well.

The ClO₂ can also be added to the transfer lines connecting the many vessels used in the starting material preparation process, the raw sugar production process and/or the overall fermentation process. Chlorine dioxide can be added in an effective amount. As one example, chlorine dioxide dosages of about 1 to about 20 mg/L can be applied to the transfer lines.

Chlorine dioxide can also be added prior to the heat exchangers at the distillery on the thin juice line to prevent bio-film formation and reduce bacteria that may be remaining in the thin juice feed. Chlorine dioxide can also be injected at the heat exchanger on each fermenter to keep bacterial counts low as the fermenters allow for a bacterial breeding area.

A side product of sugar production and/or sugar-based fermentation is vinasse. It can be used as a feed supplement. Chlorine dioxide can also be injected into the vinasse to keep bacterial counts low as this stream is another area where bacteria have a chance to increase and further infect the process.

The ability of ClO₂ to attain or surpass the efficiency of antibiotics as an antimicrobial agent is a benefit of the current method. Numerous problems accompany the use of antibiotics as microbial agents in fermentation process. Antibiotics are expensive and are not effective against all strains of bacteria.

In addition, there are other issues to consider when using antibiotics. Calculating the correct dosage of antibiotic can be a daunting task. Even after dosages have been determined, mixtures of antibiotics should be constantly or at least frequently balanced and changed in order to avoid single uses that will lead to antibiotic-resistant strains. The use of ClO₂ as an antimicrobial agent offers manufacturers a valuable option to antibiotics.

Another advantage of using ClO₂ as opposed to antibiotics deals with reduction byproducts. The ClO₂ reduces to form chlorite ion and then further reduces to form chloride ion and/or salt. The reduction from ClO₂ to chloride ion happens quickly and is indeterminate compared to the background residual already present. The chloride ion is a non-hazardous byproduct unlike those created by many antibiotics. Studies to date have shown that chloride ion does not pose a significant adverse risk to human health.

Since ClO₂ gas can decompose explosively, it is typically produced on-site. There are a number of methods of producing ClO₂ gas having a known purity, which are known to persons familiar with the technology involved here. One or more of these methods can be used. ClO₂ gas can be produced using electrochemical cells and a sodium chlorite or sodium chlorate solution. An equipment based sodium chlorate/hydrogen peroxide method also exists. Alternatively, non-equipment based binary, multiple precursor dry or liquid precursor technologies can be used. Examples of non-equipment based methods of ClO₂ generation include dry mix chlorine dioxide packets that include both a chlorite precursor packet and an acid activator packet. Other such processes include, but are not limited to, acidification of sodium chlorite, oxidation of chlorite by chlorine, oxidation of chlorite by persulfate, use of acetic anhydride on chlorite, use of sodium hypochlorite and sodium chlorite, use of dry chlorine/chlorite, reduction of chlorates by acidification in the presence of oxalic acid, reduction of chlorates by sulfur dioxide, and the ERCO R-2®, R-3®, R-5®, R-8®, R-10® and R-11® processes, from which ClO₂ is generated from NaClO₃ in the presence of NaCl and H₂SO₄ (R-2 and R-3 processes), from NaClO₃ in the presence of HCl (R-5 process), from NaClO₃ in the presence of H₂SO₄ and CH₃OH(R-8 and R-10 processes), and from NaClO₃ in the presence of H₂O₂ and H₂SO₄ (R-11 process).

Here, three methods will illustrate some possibilities. In the first method, chlorine reacts with water to form hypochlorous acid and hydrochloric acid. These acids then react with sodium chlorite to form chlorine dioxide, water and sodium chloride. In a second method, sodium hypochlorite is combined with hydrochloric or other acid to form hypochlorous acid. Sodium chlorite is then added to this reaction mixture to produce chlorine dioxide. The third method combines sodium chlorite and sufficient hydrochloric acid. In one embodiment the ClO₂ gas produced is between 0.0005 and 5.0% by weight in air.

The ClO₂ gas is dissolved in a solvent in order to create a ClO₂ solution. ClO₂ gas is readily soluble in water. In one embodiment the water and ClO₂ gas are combined in quantities that create an effective solution for application during the milling/crush of the starting material, as one example a concentration of about 20 to about 80 mg/L. In another embodiment the water and ClO₂ gas are combined in quantities that create an effective solution for application to the thin juice, for example concentrations of about 20 to about 80 mg/L or about 10 to about 50 mg/L. In another embodiment the water and ClO₂ gas are combined in quantities that create an effective solution for application during the sugar juice treatment steps to either the thin juice or the thick juice, as one example a concentration of about 10 to about 50 mg/L. In another embodiment the water and ClO₂ gas are combined in quantities that create an effective solution for application to the evaporators, vacuum pans or crystallizers used during the raw sugar production process, as one example a concentration of about 2 to about 50 mg/L. In another embodiment the water and ClO₂ gas are combined in quantities that create an effective solution for application directly into the fermentation mixture, as one example a concentration of about 2 to about 30 mg/L. In another embodiment the water and ClO₂ gas are combined in quantities that create an effective solution for application during propagation and/or conditioning, as one example a concentration of about 10 to about 85 mg/L. In another embodiment the water and ClO₂ gas are combined in quantities that create an effective solution for application to the desirable microorganism recycle tank, as one example a concentration of about 10 to about 85 mg/L. In another embodiment the water and ClO₂ gas are combined in quantities that create an effective solution for application to the yeast separator and/or centrifuge, as one example a concentration of about 10 to about 85 mg/L. In another embodiment the water and ClO₂ gas are combined in quantities that create an effective solution for application to the beer well, as one example a concentration of about 2 to about 40 mg/L. In another embodiment the water and ClO₂ gas are combined in quantities that create an effective solution for application to the transfer lines, as one example a concentration of about 1 to about 20 mg/L. In the solution of one embodiment the ClO₂ solution has an efficiency as ClO₂ in the stream of at least about 90%.

Pure or substantially pure ClO₂ is desirable because it allows the user to precisely maintain the amount of ClO₂ added to the yeast. (The single team “pure” will be used hereinafter to mean either pure or substantially pure.) If too little ClO₂ is added the dosage will not be effective in killing undesirable microorganisms. If too much ClO₂ is added it can kill the desirable yeast. If either of these situations occurs, the addition of ClO₂ will not result in more efficient ethanol production. Addition of pure ClO₂ allows the user to carefully monitor and adjust the amount of ClO₂ added to the yeast. This enables the user to add adequate ClO₂ to improve microbial efficacy without killing the yeast.

The ClO₂ solution is introduced at some point during the production of ethanol or sugar. The ClO₂ solution can be added in the starting material preparation process, the raw sugar production process and/or the overall fermentation process. The ClO₂ solution can be added to milling vessels, thick juice treatment vessels, thin juice treatment vessels, vacuum pans, sugar crystallizers, evaporators, transfer lines, yeast recycle tanks, yeast separators, centrifuges, beer wells, cook vessels, fermentation tanks, propagation tanks, conditioning tanks, starter tanks or tanks used during liquefaction. The ClO₂ solution can also be added to the piping between these units or heat exchangers.

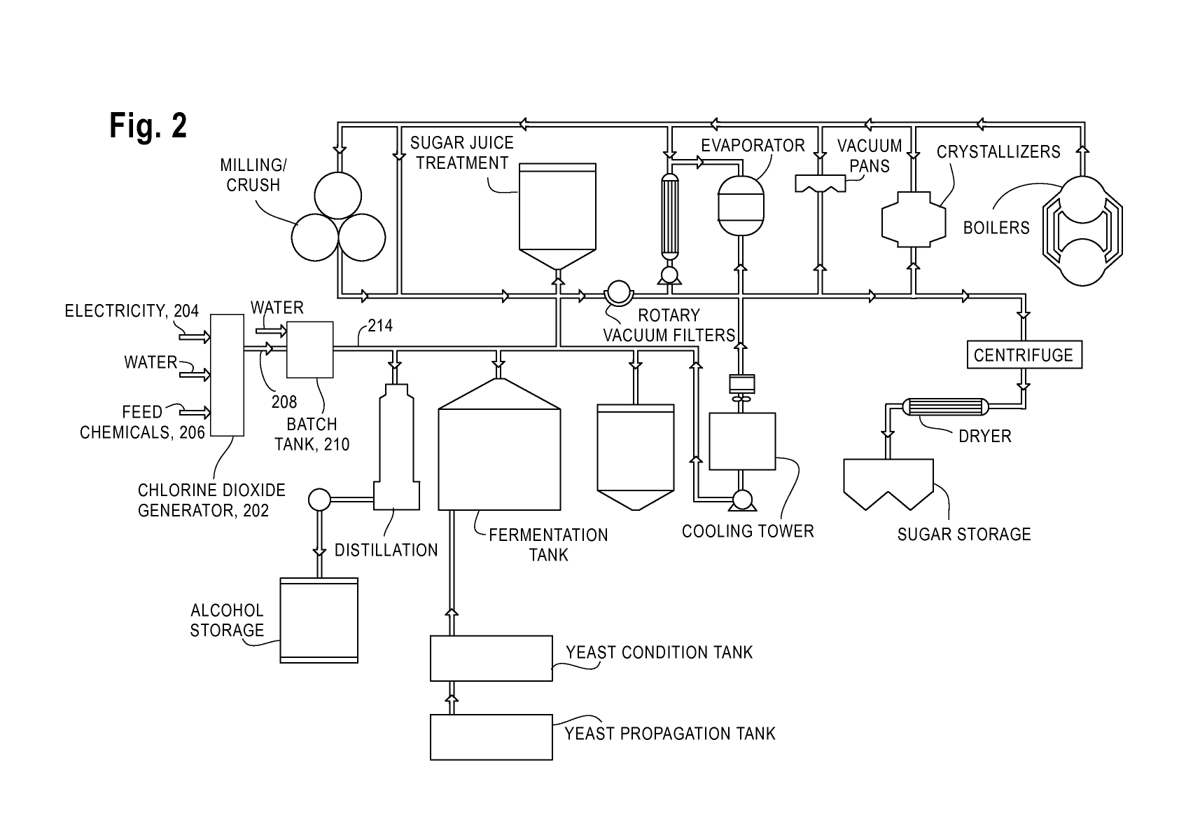

FIG. 2 illustrates an apparatus for carrying out the fermentation process with an integrated ClO₂ system. The apparatus has a ClO₂ generator. The ClO₂ generator has an input for electricity. There is also an inlet for at least one chlorine containing chemical. There are three different types of chemical feed systems: a vacuum system, a pressure system and a combination system. Many types of feed systems can be employed to deliver chemicals in a fluid state. Chlorine gas, for example, can be added by a vacuum or combination feed system. The ClO₂ generator should also have an outlet for exhausting a ClO₂ gas stream from the generator. In one embodiment the ClO₂ gas stream exiting the generator is between 0.0005 and 5.0% by weight in air.

A batch tank that receives the ClO₂ gas stream is fluidly connected to the ClO₂ generator outlet. In the batch tank the ClO₂ gas is dissolved in water to form a ClO₂ solution. The batch tank has an inlet for introducing a water stream. The water stream and the ClO₂ gas stream are combined to form a ClO₂ solution. The concentration of the ClO₂ solution in the batch tank can vary across a wide range. Concentrations of up to about 5,000 mg/L can be achieved and concentrations of up to about 8,000 mg/L can be achieved with additional equipment. The ClO₂ solution is then exhausted from the batch tank through an outlet at a specified dosage rate to create a solution of the desired concentration. In one embodiment the dosed ClO₂ solution, for application during the milling/crush of the starting material has an effective concentration, as one example about 20 to about 80 mg/L. In another embodiment the dosed ClO₂ solution, for application to the thin juice has an effective concentration, for example about 20 to about 80 mg/L or about 10 to about 50 mg/L. In another embodiment the dosed ClO₂ solution, for application during the sugar juice treatment steps to either the thin juice or the thick juice has an effective concentration, as one example about 10 to about 50 mg/L. In another embodiment the dosed ClO₂ solution, for application to the evaporators, vacuum pans or crystallizers used during the raw sugar production process has an effective concentration, as one example about 2 to about 50 mg/L. In another embodiment the dosed ClO₂ solution, for application directly into the fermentation mixture has an effective concentration, as one example about 2 to about 30 mg/L. In another embodiment the dosed ClO₂ solution, for application during propagation and/or conditioning has an effective concentration, as one example about 10 to about 85 mg/L. In another embodiment the dosed ClO₂ solution, for application to the desirable microorganism recycle tank has an effective concentration, as one example about 10 to about 85 mg/L. In another embodiment the dosed ClO₂ solution, for application to the yeast separator and/or centrifuge has an effective concentration, as one example about 10 to about 85 mg/L. In another embodiment the dosed ClO₂ solution, for application to the beer well has an effective concentration, as one example about 2 to about 40 mg/L. In another embodiment the dosed ClO₂ solution, for application to the transfer lines has an effective concentration, as one example about 1 to about 20 mg/L. In one embodiment, the exiting ClO₂ solution has an efficiency as ClO₂ in the stream of at least about 90%.

A production vessel is fluidly connected to the batch tank via the ClO₂ solution outlet. The production vessel could be a milling vessel, thick juice treatment vessel, thin juice treatment vessel, vacuum pan, sugar crystallizer, evaporator, transfer line, yeast recycle tank, yeast separator, centrifuge, beer well, cook vessel, fermentation tank, propagation tank, conditioning tank, starter tank or tank used during liquefaction. Multiple production vessels could be fluidly connected to a single batch tank, as shown in FIG. 2. Introducing the ClO₂ solution into the production vessel is capable of decreasing the concentration of undesirable microorganisms and potentially also promoting propagation of yeast present.

### EXAMPLE 1

A thin juice line at a sugar plant that fed into a distillery for fermentation was treated with chlorine dioxide according to the present method. Previously the thin juice line had been treated using sulfuric acid to decrease the pH. The trial evaluated the bacterial efficacy of chlorine dioxide at an elevated pH in order to reduce sulfuric acid use without causing a detrimental effect to the yeast or fermentation.

In the trial, 90% pure chlorine dioxide solution was generated. Twenty parts per million (ppm) of chlorine dioxide was introduced into the thin juice line. Thin juice samples were collected before during and after the trial. The samples were examined for chlorite, chlorate and chloride residuals. The results show that chlorite and chlorate residuals were not detected and chloride concentrations were within the same range as baseline. This indicates that byproducts from chlorine dioxide in the thin juice are not a concern.

During the trial, bacterial samples were also collected and analyzed at various locations in the system. The results are shown in the table below.

TABLE 1
Lactate Concentration (g/L) at
Five Locations in the Fermentor Over Thirty Seven Days

| Day of | First | Second | Third | Fourth | Fifth |
|---|---|---|---|---|---|
| Trial | location | location | location | location | location |
| 1 | 0.41 | 0.56 | 0.61 | 0.52 | 0.62 |
| 2 | 0.45 | 0.57 | 0.57 | 0.6 | 0.57 |
| 3 | 0.36 | 0.62 | 0.6 | 0.64 | 0.65 |
| 4 | 0.39 | 0.6 | 0.6 | 0.58 | 0.6 |
| 5 | 0.44 | 0.66 | 0.71 | 0.69 | 0.75 |
| 6 | 0.39 | 0.54 | 0.62 | 0.71 | 0.69 |
| 7 | 0.51 | 0.76 | 0.81 | 0.88 | 0.9 |
| 8 | 0.44 | 0.55 | 0.64 | 0.7 | 0.75 |
| 9 | 0.5 | 0.68 | 0.74 | 0.78 | 0.83 |
| 10 | 0.55 | 0.77 | 0.82 | 0.83 | 0.84 |
| 11 | 0.4 | 0.5 | 0.55 | 0.65 | 0.8 |
| 12 | | | | | |
| 13 | 0.22 | 0.33 | 0.36 | 0.39 | 0.42 |
| 14 | 0.22 | 0.36 | 0.37 | 0.4 | 0.51 |
| 15 | 0.22 | 0.32 | 0.34 | 0.4 | 0.5 |
| 16 | 0.3 | 0.4 | 0.45 | 0.5 | 0.6 |
| 17 | 0.29 | 0.43 | 0.5 | 0.57 | 0.67 |
| 18 | 0.43 | 0.48 | 0.54 | 0.6 | 0.62 |
| 19 | 0.4 | 0.6 | 0.7 | 0.8 | 1.1 |
| 20 | 0.55 | 0.7 | 0.75 | 0.5 | 0.85 |
| 21 | 0.9 | 1.16 | 1.24 | 1.35 | 1.36 |
| 22 | 1.31 | 1.73 | 2.01 | 2.04 | 1.66 |
| 23 | 1.39 | 1.75 | 2.01 | 2.19 | 2.15 |
| 24 | 1.12 | 1.41 | 1.73 | 2 | 2.13 |
| 25 | 0.94 | 1.35 | 1.6 | 1.82 | 1.97 |
| 26 | 0.81 | 1.08 | 1.34 | 1.57 | 1.75 |
| 27 | 0.56 | 0.62 | 0.77 | 0.94 | 1.18 |
| 28 | 0.49 | 0.53 | 0.66 | 0.63 | 0.82 |
| 29 | 0.49 | 0.56 | 0.68 | 0.8 | 0.87 |
| 30 | 1.25 | 1.27 | 1.33 | 1.25 | 1.13 |
| 31 | 1.33 | 1.95 | 2.24 | 2.33 | 1.98 |
| 32 | 1.82 | 2.32 | 2.52 | 2.57 | 2.49 |
| 33 | 1.69 | 2.33 | 2.56 | 2.7 | 2.45 |
| 34 | 2.33 | 3.06 | 3.21 | 3.1 | 2.97 |
| 35 | 1.82 | 2.56 | 3.06 | 3.33 | 3.56 |
| 36 | 1.77 | 2.34 | 2.72 | 2.89 | 3.07 |
| 37 | 1.67 | 2.21 | 2.43 | 2.65 | 2.83 |

The data indicates a low level of lactate before chlorine dioxide was introduced at the normal pH of 3.5. The lactate trended downward once the chlorine dioxide treatment was started at 20 ppm with a pH elevation to 4. On day 18 the equipment had to be shutdown due to an unrelated issue. The data shows that the lactate level never recovered after the shutdown.

This data demonstrates that chlorine dioxide treatment can effectively treat bacteria in a thin juice line. Addition of substantially pure chlorine dioxide solution can significantly reduce bacteria in a thin juice line. This provides significant savings in sulfuric acid expenditures by cutting sulfuric acid use, provide a better environment for the yeast and increases overall ethanol yield. Additional injection points in the distillery would improve bacteria reduction even further.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

What is claimed is:

1. A method of reducing undesirable microorganism concentration in an aqueous fluid stream employed in a sugar production process, the method comprising the steps of:
(a) employing an aqueous fluid stream in a sugar production process;
(b) generating ClO₂ gas;
(c) dissolving said ClO₂ gas to form a ClO₂ solution;
(d) introducing an aqueous ClO₂ solution into said stream.

2. The method of claim 1 wherein said steps are performed sequentially.

3. The method of claim 1 wherein said ClO₂ solution has a concentration between about 20 and about 80 mg/L.

4. The method of claim 1 wherein said ClO₂ solution has a concentration between about 10 and about 50 mg/L.

5. The method of claim 1 wherein said ClO₂ solution has a concentration between about 2 and about 50 mg/L.

6. The method of claim 1 wherein said ClO₂ solution has an efficiency as ClO₂ in the stream of at least 90%.

7. A method of reducing undesirable microorganism concentration in an aqueous fluid stream employed in a sugar production process, the method comprising the steps of:
(a) employing an aqueous fluid stream in a sugar production process; and
(b) introducing ClO₂ having an efficiency as ClO₂ of at least 90% into said stream.

8. The method of claim 7 wherein said steps are performed sequentially.

9. The method of claim 7 wherein said ClO₂ solution has a concentration between about 20 and about 80 mg/L.

10. The method of claim 7 wherein said ClO₂ solution has a concentration between about 10 and about 50 mg/L.

11. The method of claim 7 wherein said ClO₂ solution has a concentration between about 2 and about 50 mg/L.

12. The method of claim 7 wherein said ClO₂ is a gas.

13. An apparatus for reducing undesirable microorganism concentration employed in a sugar production process, the apparatus comprising:
(a) a ClO₂ generator comprising an inlet for introducing at least one chlorine-containing feed chemical and an outlet for exhausting a ClO₂ gas stream from said generator;
(b) a batch tank fluidly connected to said ClO₂ generator outlet, said batch tank receiving said ClO₂ gas stream from said ClO₂ generator outlet, said batch tank comprising an inlet for introducing a second water stream and an outlet for exhausting an aqueous ClO₂ solution from said batch tank;
(c) a vessel utilized in a sugar-production process, said vessel fluidly connected to said batch tank;
wherein introducing said ClO₂ solution from said batch tank to said vessel reduces undesirable microorganism concentration in said vessel.

14. The apparatus of claim 13 wherein said vessel is a milling vessel.

15. The apparatus of claim 13 wherein said vessel is a thin juice treatment vessel.

16. The apparatus of claim 13 wherein said vessel is a thick juice treatment vessel.

17. The apparatus of claim 13 wherein said vessel is an evaporator.

18. The apparatus of claim 13 wherein said vessel is a vacuum pan.

19. The apparatus of claim 13 wherein said vessel is a crystallizer.

20. The apparatus of claim 13 wherein said aqueous ClO₂ solution exhausted from said batch tank is dosed to a concentration between about 20 and about 80 mg/L.

21. The apparatus of claim 13 wherein said aqueous ClO₂ solution exhausted from said batch tank is dosed to a concentration between about 10 and about 50 mg/L.

22. The apparatus of claim 13 wherein said aqueous ClO₂ solution exhausted from said batch tank is dosed to a concentration between about 2 and about 50 mg/L.

23. A method of reducing undesirable microorganism concentration, promoting yeast propagation/conditioning, and increasing yeast efficiency in an aqueous fluid stream employed in a sugar-based fermentation process, the method comprising the steps of:
(a) introducing a quantity of fermentable sugar to said stream;
(b) introducing a quantity of yeast to said stream;
(c) generating ClO₂ gas;
(d) dissolving said ClO₂ gas to form a ClO₂ solution;
(e) introducing an aqueous ClO₂ solution into said stream.

24. A method of reducing undesirable microorganism concentration, promoting yeast propagation/conditioning, and increasing yeast efficiency in an aqueous fluid stream employed in a sugar-based fermentation process, the method comprising the steps of:
(a) introducing a quantity of fermentable sugar to said stream;
(b) introducing a quantity of yeast to said stream; and
(c) introducing ClO₂ having an efficiency as ClO₂ of at least 90% into said stream.

25. An apparatus for reducing undesirable microorganism concentration, promoting yeast propagation/conditioning, and increasing yeast efficiency employed in a sugar-based fermentation process, the apparatus comprising:
(a) a ClO₂ generator comprising an inlet for introducing at least one chlorine-containing feed chemical and an outlet for exhausting a ClO₂ gas stream from said generator;
(b) a batch tank fluidly connected to said ClO₂ generator outlet, said batch tank receiving said ClO₂ gas stream from said ClO₂ generator outlet, said batch tank comprising an inlet for introducing a second water stream and an outlet for exhausting an aqueous ClO₂ solution from said batch tank;
(c) a vessel for containing an aqueous yeast solution, said vessel fluidly connected to said batch tank;
wherein introducing said ClO₂ solution from said batch tank to said vessel promotes propagation of yeast present in said vessel.
